(19) United States
(12) Patent Application Publication
(10) Pub. No.: US 2025/0277165 A1
(43) Pub. Date: Sep. 4, 2025

(54) BIOPOLYOLS AND BIOPOLYMERS AND THEIR APPLICATIONS

(71) Applicant: NUOL Green Chemistry LLC, Little Falls, MN (US)

(72) Inventor: Setsuo Sato, Piracicaba (BR)

(21) Appl. No.: 18/596,160

(22) Filed: Mar. 5, 2024

(30) Foreign Application Priority Data
Mar. 1, 2024 (BR) 10 2024 004253 0

(51) Int. Cl. C11C 3/00; C12N 9/20; C12P 7/6436

(52) U.S. Cl. CPC C11C 3/003; C12N 9/20; C12P 7/6436; C12Y 301/01003

(57) ABSTRACT

A family of polyols with superior functionality to existing polyols of both vegetable and petrochemical origin are provided. The greater functionality provides much higher performance due to the greater number of hydroxyls per weight of product. Therefore, biopolymers produced through these biopolyols have superior performance and a more advantageous cost. In addition to performance and economic aspects, the environmental benefits are enormous due to the non-generation of waste, products are completely biodegradable, non-toxic, non-corrosive, harmless, waste such as frying oil, residual fatty acids can be used. The key and crucial point of the products is to obtain a starting material with a high content of unsaturated fatty chains, that is, with an oleic and linoleic chain content and a low amount of stearic and palmitic acid.

Biopolymers based on vegetable oil derivatives are one of the most investigated classes currently due to their numerous benefits. Four classes of polymers Polyurethanes, polyesters, polyolefins and poly ethers are the targets for the use of these products. For this to become a reality, it is necessary to have biopolyols with better performance than polyols of petrochemical origin and that are more economically advantageous.

In this development we created a family of polyols with superior functionality to existing polyols of both vegetable and petrochemical origin. The greater functionality provides much higher performance due to the greater number of hydroxyls per weight of product. Therefore, the biopolymers produced through these biopolyols have superior performance and a more advantageous cost.

In addition to performance and economic aspects, the environmental benefits are enormous due to the non-generation of waste, products are completely biodegradable, non-toxic, non-corrosive and harmless. Waste such as frying oil and residual fatty acids can be used.

The key and crucial point of the products is to obtain a starting material with a high content of unsaturated fatty chains, that is, with a minimum amount of stearic and palmitic acid.

### STATUS QUO

Without mentioning polyols of petrochemical origin, those produced from castor oil are the most common from a commercial point of view and published in scientific articles as demonstrated in the two chemical structures in FIGS. 1 and 2.

The structural characteristic, flexibility, green chemistry of this product group have contributed greatly to the development of this “RICINOLEICA” platform, the reality is that the availability of castor oil will not be sufficient in the near future.

Therefore, it is very important in the development of new materials to support the necessary volumes of materials with a high degree of unsaturation.

### DESCRIPTION OF THE INVENTION

The chemical structures demonstrated in FIGS. 3 and 4 demonstrate the versatility of molecules created according to the required applications and the ways of managing the costs of each product.

In this way, the following are described:

1. Production of the chemical structures in FIGS. 3 to 5, with the aforementioned chemical structures being products synthesized from fatty materials with a high unsaturated content composed of oleic and linoleic acids, and from these fatty acids, their respective monoesters, diesters and triesters.

2. Production, in accordance with item 1, wherein after the esterification process, the products are epoxidized using hydrogen peroxide and performic acid as catalyst with a minimum conversion of 80% into oxirane rings.

3. Production, according to any of items 1 to 2, wherein from esters with a high content of oxirane rings, the epoxide rings are opened with methanol, ethanol, isopropanol, butanol, isobutanol, isopentanol, cyclohexanol, ethylhexanol, decanol, dodecanol, glycerin, neopentyl glycol, trimethylolpropane, pentaerythritol, polyglycerin-3, polyglycerin-6.

4. Production, in accordance with any of items 1 to 3, comprising the following steps:
selection of vegetable oils and their respective fatty acids with an iodine index above 100 cg 12/100 grams of product (soy, corn, peanut, sunflower, rapeseed, canola, palm);
enzymatic hydrolysis using lipases at a temperature of 40° C. with a minimum conversion of 97% in the case of vegetable oils;
selective crystallization with separation of the saturated chains, with the unsaturated phase containing a maximum of 6% of saturated material (stearic and palmitic);
esterification of fatty acids with alcohols at a temperature of 80° C. to 240° C. with a minimum 99% conversion (Methanol, ethanol, isopropanol, butanol, isobutanol, cyclohexanol, ethyl hexanol, decanol, dodecanol);
epoxidation of the fatty esters of item 4 via hydrogen peroxide catalyzed by performic acid with conversion of the unsaturated chains at least 80%; and
opening of epoxy rings with methanol, ethanol, isopropanol, butanol, isobutanol, isopentanol, cyclohexanol, ethylhexanol, decanol, dodecanol, glycerin, neopentylglycol, trimethylolpropane, pentaerythritol, polyglycerin-3, polyglycerin-6.

5. Use of products produced in accordance with items 1 to 4 for the production of polyurethanes, polyesters, polyethers, and polyolefins.

6. Biopolyols obtained in accordance with items 1 to 4, in which biopolyols contribute to the reduction of carbon emissions, are biodegradable, non-toxic, safe to handle and can be made from waste materials originating in the food, biodiesel industries and alcoholic.

### BRIEF DESCRIPTION OF THE FIGURES:

FIG. 1 represents the chemical structure of the biopolyol derived from castor oil from the prior art.

FIG. 2 represents the chemical structure of hydroxylated castor oil from the prior art.

FIG. 3 represents the chemical structure of a hydroxylated synthetic vegetable oil according to the invention.

FIG. 4 represents the chemical structure of a hydroxylated synthetic vegetable oil according to the invention.

FIG. 5 represents the chemical structure of a hydroxylated fatty acid ester according to the invention.

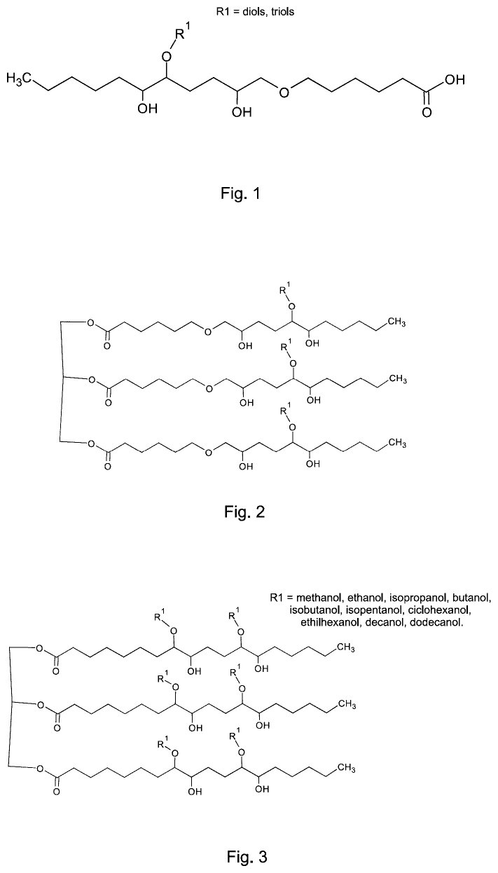

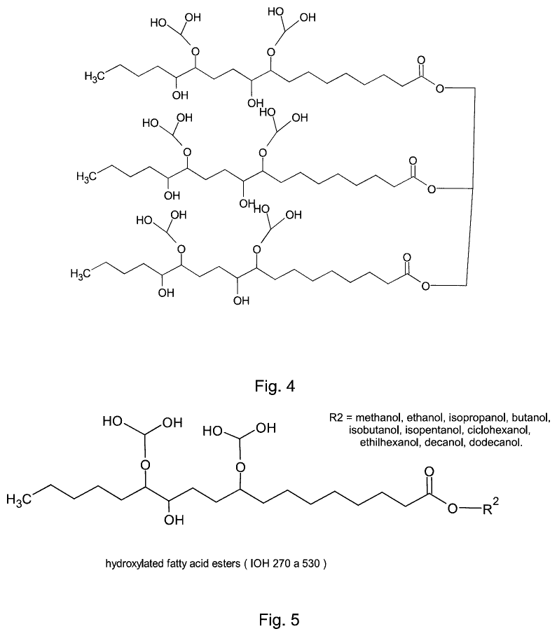

### STEPS USED IN PRODUCT PREPARATION

1. Selection of vegetable oils and their respective fatty acids with an iodine index above 100 cg 12/100 grams of product (soy, corn, peanut, sunflower, rapeseed, canola, palm).

2. Enzymatic hydrolysis using lipases at a temperature of 40° C. with a minimum conversion of 97% in the case of vegetable oils.

3. Selective crystallization with separation of saturated chains, with the unsaturated phase containing a maximum of 6% saturated material (stearic and palmitic).

4. Esterification of fatty acids with alcohols at a temperature of 80° C. to 240° C. with a minimum 99% conversion (Methanol, ethanol, isopropanol, butanol, isobutanol, cyclohexanol, ethyl hexanol, decanol, dodecanol).

5. Epoxidation of fatty esters from item 4 via hydrogen peroxide catalyzed by performing acid with conversion of unsaturated chains at least 80%.

6. Opening of epoxy rings with methanol, ethanol, isopropanol, butanol, isobutanol, isopentanol, cyclohexanol, ethylhexanol, decanol, dodecanol, glycerin, neopentylglycol, trimethylolpropane, pentaerythritol, polyglycerin-3, polyglycerin-6.

1. Production of biopolyols characterized in that said chemical structures are products synthesized from fatty materials with a high unsaturated content composed of oleic and linoleic acids, and from these fatty acids, their respective monoesters, diesters and triesters are produced.

2. Production, according to claim 1, characterized in that, after the esterification process, the products are epoxidized using hydrogen peroxide and performic acid as catalyst with a minimum conversion of 80% into oxirane rings.

3. Production, according to claim 1, characterized in that, from esters with a high content of oxirane rings, the epoxide rings are opened with methanol, ethanol, isopropanol, butanol, isobutanol, isopentanol, cyclohexanol, ethylhexanol, decanol, dodecanol, glycerin, neopentyl glycol, trimethylolpropane, pentaerythritol, polyglycerin-3, polyglycerin-6.

4. Production, according to claim 1, characterized in that it comprises the following steps:
selection of vegetable oils and their respective fatty acids with an iodine index above 100 cg 12/100 grams of product (soy, corn, peanut, sunflower, rapeseed, canola, palm);
enzymatic hydrolysis using lipases at a temperature of 40° C. with a minimum conversion of 97% in the case of vegetable oils;
selective crystallization with separation of the saturated chains, with the unsaturated phase containing a maximum of 6% of saturated material (stearic and palmitic);
esterification of fatty acids with alcohols at a temperature of 80° C. to 240° C. with a minimum 99% conversion (Methanol, ethanol, isopropanol, butanol, isobutanol, cyclohexanol, ethyl hexanol, decanol, dodecanol);
epoxidation of the fatty esters of item 4 via hydrogen peroxide catalyzed by performic acid with conversion of the unsaturated chains at least 80%; and
opening of epoxy rings with methanol, ethanol, isopropanol, butanol, isobutanol, isopentanol, cyclohexanol, ethylhexanol, decanol, dodecanol, glycerin, neopentylglycol, trimethylolpropane, pentaerythritol, polyglycerin-3, polyglycerin-6.

5. Use of products produced according to claim 1 characterized in that it is for the production of polyurethanes, polyesters, polyethers and polyolefins.

6. Biopolyols characterized in that they contribute to the reduction of carbon emissions, are biodegradable, non-toxic, safe to handle and can be made from waste materials originating in the food, biodiesel and alcohol industries.
